(19) United States
(12) Patent Application Publication
(10) Pub. No.: US 2017/0135692 A1
(43) Pub. Date: May 18, 2017

(54) SYSTEMS AND METHODS FOR HELICALLY ADVANCING SUTURE IN TISSUE

(71) Applicant: VasoStitch, Inc., Santa Cruz, CA (US)

(72) Inventors: Amir Belson, Cupertino, CA (US); Luke Clauson, Redwood City, CA (US); Michael Schaller, Redwood City, CA (US)

(73) Assignee: VasoStitch, Inc., Santa Cruz, CA (US)

(21) Appl. No.: 15/419,971

(22) Filed: Jan. 30, 2017

Related U.S. Application Data
(63) Continuation of application No. PCT/US2015/043312, filed on Jul. 31, 2015.
(60) Provisional application No. 62/031,694, filed on Jul. 31, 2014.

(51) Int. Cl. A61B 17/04; A61B 17/06; A61B 17/00

(52) U.S. Cl. CPC A61B 17/0469; A61B 17/0057; A61B 17/0401; A61B 17/06166; A61B 2017/00663; A61B 2017/0427; A61B 2017/0406; A61B 2017/00247; A61B 2017/00575; A61B 2017/0437; A61B 2017/0462; A61B 2017/0464; A61B 2017/06171

(57) ABSTRACT

Systems and methods for providing transapical access to a heart chamber for performing an intra cardiac procedure are described. The systems include a helical needle driver and a dilator. The helical needle driver rotates and translates a shuttle member which advances one or more helical needles to place a helical suture within the myocardium. After removing the needles, the dilator is advanced through the pre-placed helical suture, dilating both a passage and the circumscribing suture. After performing procedure, the pre-placed suture may be closed by proximally retracting an external end of the suture.

### CROSS-REFERENCE APPLICATIONS

This application is a continuation of PCT Application No. PCT/US2015/043312 (Attorney Docket No. 39277-708.601), filed Jul. 31, 2015, which claims the benefit of U.S. Provisional Application No. 62/031,694 (Attorney Docket No. 39277-708.101), filed on Jul. 31, 2014, the full disclosures of which are incorporated herein in their entirety.

### BACKGROUND OF THE INVENTION

1. Field the invention

The present invention relates generally to devices and systems for advancing and anchoring lengths of suture in tissue. More particularly, the invention relates to anchoring suture in tissue for closing penetrations through the tissue.

Sutures are commonly used by physicians for closing wounds, incisions, fistulas, and other common tissue defects. When the defects are close to a patient's skin or other tissue surface, it is usually easy for the physician to use a needle to sew the wound closed. When the defect lies well below the tissue surface, in contrast, placing sutures can be much more difficult, and a variety of tools have been developed over the years to assist in such placement. For example, numerous suturing tools have been developed for closing penetrations in the femoral artery following angioplasty and other intravascular procedures. The tools typically include a shaft which is advanced through a tissue tract which is formed through the patient's thigh to reach the femoral artery. The tools are manipulated to place the suture over a proximal opening of the penetration, and the physician then tensions the suture to close the remote opening through the femoral wall.

While such remote suturing tools have been very successful for femoral artery closure and other purposes (such as closing laparoscopic wounds), and have allowed procedures that were not previously possible, the use of the remote suturing tools still suffers from certain limitations. For example, in many cases it is necessary to both introduce the suture through a long tissue tract and to subsequently draw the opposite end of the tissue up through the same tract. Once the tissue is in place, it can be difficult to control the tension being placed on the suture to close the remote wound. In particular, inexperienced physicians can either supply insufficient tension, in which the wound does not fully close, or apply too much tension which can either break the suture or unnecessarily damage tissue surrounding the wound. Finally, the need to tie off the suture in the vicinity of the remote wound can also be very challenging.

Of particular interest to the present invention, commonly owned US 2012/0116418 describes a helical needle advancement device for placing sutures prior to forming a transapical tissue tract using a needle and a dilator for access to a patient's heart chamber. While a highly effective and efficient design, the direct linkage between the driving knob and the needles can limit the operability in certain circumstances.

For these reasons, it would be desirable to provide improved methods and systems for the advancement and anchoring of suture in tissue, particularly in procedures where remote or inaccessible wounds are being sutured. It would be particularly desirable to provide methods and tools which facilitate advancing a length of suture within solid tissue and optionally anchoring a distal end of the suture length at a remote location in the tissue. At least of these objectives will be met by the inventions described below.

2. Description of the Background Art

Commonly owned US 2012/0116418 has been described above. Other commonly owned patents and applications which are relevant to remote suturing include U.S. Pat. No. 9,078,633; US 2015/0073478; and US 2012/035654. The full disclosures of each of these commonly owned patents and publications are incorporated herein by reference. Other patents and publications of interest include U.S. Pat. Nos. 8,500,757; 6,626,917; 6,287,250; and 5,577,993; and U.S. Patent Publication Nos. 2011/0238090; 2011/0190811; and 2006/0074484.

### SUMMARY OF THE INVENTION

The present invention provides improved methods and systems for advancing, anchoring, and tensioning suture and tissue. While particularly useful for forming, accessing, and closing transapical tissue tracts as well as closing wounds, incisions, fistulas, and the like, the present invention will be useful in any procedure where a length of suture is advanced into tissue, a distal end of the suture anchored at a remote location within the tissue, and a proximal end of the suture pulled or otherwise tensioned to close a remote wound or otherwise perform a remote tissue manipulation.

In other aspects, the present invention provides a pledget which allows “auto-locking” and cinching of the suture on the tissue surface. The pledget includes a mechanism which allows suture to pass in one direction only which allows convenient cinching by pulling on a free end of the suture when closing the tissue tract. The present invention further provides self-deploying tissue anchors which are attached at a distal end of a suture length and which can be advanced distally through tissue with minimal force but which firmly anchor in tissue when the suture length is retracted. The present invention still further provides a needle-dilator device for creating a tissue tract where the needle is provided with a latch mechanism to prevent over-insertion and unintended puncturing of tissue.

In one aspect, the present invention provides systems for helically advancing suture through tissue for any of the procedures listed above. The system comprises a handle having a distal end, a proximal end, and a central passage extending between the distal end and the proximal ends. A knob is rotatably carried on the proximal end of the handle, and a shuttle member is reciprocatably disposed in the central passage of the handle. The shuttle member also has a distal end, a proximal end, and a central passage extending between the distal end and the proximal end. At least one helical needle is coupled to the distal end of the shuttle member, and the helical needle(s) releasably carries a length of suture which is intended to be deployed within a target tissue. In a specific feature of the present invention, the knob is coupled to the shuttle so that rotation of the knob rotates and axially translates the shuttle member in order to rotate and translate the at least one helical needle. The knob is coupled to the shuttle in such a way that the knob itself will not axially translate as it is rotated but will be still be able to impart such axial translation to the shuttle member.

The systems of the present invention will often further comprise a needle-dilator assembly which is adapted or configured to be received through the central passage of the shuttle member, and the needle-dilator assembly will usually pre-mounted in the central passage of the shuttle member, where the resulting assembly can be packaged and sterilized as a unit or an assembly available for immediate use. Packaging may be accomplished in any conventional medical device package including a bag, box, tube, or the like, where sterilization may be performed before and/or after sealing in the package. In the package configuration, a straight needle of the needle-dilator assembly is typically positioned to extend distally from a distal end of the central passage of the shuttle member while a dilator body of the needle-dilator assembly remains retracted within the central passage of the shuttle member.

In preferred embodiments, the straight needle of the needle-dilator assembly will be latched to the handle so that the dilator body of the needle-dilator assembly may be advanced over the straight needle without advancing the needle. The straight needle will automatically unlatch (requiring no action by the user other than advancing the dilator body over the needle) when the dilator is fully advanced over the straight needle so that the straight needle and/or needle-dilator assembly can be withdrawn from the central passage of the shuttle member. The ability to latch the needle to prevent unintended advancement reduces the possibility that the needle will unintentionally penetrate tissue outside of a target site. In specific embodiments, the latching mechanism of the needle-dilator assembly comprises a spring-loaded detent which travels over a cam surface which moves in unison with the dilator body. The detent engages the handle (thus immobilizing the needle relative to the handle of the suture deployment device) while in the package configuration (prior to dilator body advancement), and the dilator body is configured to reposition the cam surface as the dilator is advanced relative to the straight needle to allow the detent to fall out of engagement with the handle, thus allowing the needle to be withdrawn from the shuttle member after the needle is covered by the dilator body.

In other specific embodiments, the distal end of the handle may be adapted to engage and stabilize against either a myocardial surface or a pericardial of a patient's heart. The handle may be further adapted to engage an apical region of the heart either through an intercostal access site or through a subxiphoid approach.

In exemplary embodiments, the at least one helical needle is fixedly attached to the distal end of the shuttle so that the needle is advanced through tissue as the shuttle rotates and advances. In certain embodiments, the helical needle may be hollow and the suture may be carried within the needle itself. In other embodiments, the handle may have one or more pockets or receptacles disposed on an outer surface near distal end thereof, and the needle may be received in the pocket. The suture will usually be configured so that a distal end of the suture length will embed or implant in tissue, for example having barbs disposed along a distal end or region of the suture where the barbs are adapted to self-deploy to anchor in the myocardial or other tissue as the helical needle is withdrawn. In other embodiments, the suture may include non-penetrating anchors which are suitable for deployment within a target body cavity, such as the left ventricle, when the needle is advanced into the cavity.

In still other specific embodiments, the system will include two or more helical needles, usually having two needles disposed in a common cylindrical envelope with the turns of the needles being 180° out-of-phase. In still further embodiments, the two or more helical needles could have different diameters and be arranged coaxially.

The knob is coupled to the shuttle so that the rotation of the knob rotates and axially translates the shuttle member to rotate and translate the at least one helical needle. In specific embodiments, an outer surface of the shuttle and an inner surface of the central passage of the handle together define a helical track and a track follower so that rotation of the shuttle relative to the handle causes the shuttle to axially translate relative to the handle. Usually, a coupling element on the knob engages a coupling element on the shuttle so that rotation of the knob is transferred to the shuttle to cause the shuttle to axially translate in response to interaction of the helical track and the track follower without the knob being axially displaced.

In another aspect, the present invention provides a method for forming transapical access to a heart chamber, particularly a left ventricle. The method comprises positioning a distal end of a handle against a surface of the heart, typically an apex of a patient's heart, to advance a distal end of a straight needle through myocardial tissue into a heart chamber. At least one helical needle is rotated and advanced from a distal end of the handle into the myocardial tissue surrounding the needle, where the helical needle carries a length of suture. The helical needle is then reverse rotated and retracted to embed an anchor at the free end of the length of suture in the myocardial tissue and form a helical path surrounding the straight needle in the myocardial tissue. A dilator is then advanced from the handle over the straight needle within the embedded helical suture. The straight needle is latched to the handle so that the needle cannot be further advanced into the heart chamber as the dilator advances. The needle unlatches from the handle when the dilator fully covers the needle. The handle may then be removed over the dilator, and then access sheath is advanced over the dilator to provide an interventional access into the heart chamber.

In specific embodiments, the physician may observe blood flashback through the needle to confirm entry of the needle into the heart chamber. The needle will be latched to the dilator body by a spring-loaded detent that engages the handle, where advancement of the dilator body fully over the needle disengages the detent from the handle to allow the needle and/or needle-dilator assembly to move freely of the handle.

In further specific embodiments, rotating a knob relative to the handle will rotate and advance the at least one helical needle. Usually, the knob is coupled to a shuttle that carries the at least one helical needle, and rotation of the knob rotates and axially translates the shuttle member to rotate and translate the at least one helical needle. While the knob rotates, it does not axially translate relative to the handle while it is being rotated.

In other specific embodiments of the method, the method steps are performed while the heart is beating. Optionally, tension may be applied by the handle to the pericardium to stabilize the heart while the helical needle is being advanced. Alternatively, the handle may be applied directly to the epicardial surface and adhere to the surface, for example using cleats on the handle.

### BRIEF DESCRIPTION OF THE DRAWINGS

FIG. 1 illustrates an exemplary system according to the present invention for helically advancing suture and tissue and including a suture deployment device and a needle-dilator assembly.

FIG. 2 is an exploded view of the suture deployment device of FIG. 1.

FIG. 3 is an exploded view of the needle-dilator assembly of FIG. 1.

FIG. 4 is a cross-sectional view of the suture deployment device of FIGS. 1 and 2.

FIG. 5 is a detail take along line 5-5 of FIG. 4.

FIG. 6 is a cross-sectional view take along lines 6-6 of FIG. 5.

FIG. 7A is a detailed view of a distal end of the suture deployment device of FIGS. 1 and 2 shown with a needle of the needle-dilator assembly extending from said distal end as it would be configured in the pre-loaded or “packaged” configuration of the system.

FIG. 7B is a view similar to that of FIG. 7A, with a single helical needle shown in its distally advanced position. The second needle of the suture deployment device is not shown for simplicity.

FIG. 8 is a detailed, cross-sectional view of a proximal end of the suture deployment device showing a latching mechanism which holds the needle in place when the needle-dilator assembly is present in the suture deployment device.

FIGS. 9A-9C illustrate how the latching mechanism is released when the dilator body is advanced over the straight needle.

FIGS. 10A and 10B illustrate an exemplary self-deploying suture anchor configured to embed in tissue.

FIG. 11 illustrates an exemplary suture pledget used for cinching a suture externally over an access site.

FIGS. 12A and 12B illustrate a second exemplary design for an pledget.

FIGS. 13A through 13K illustrate use of the suture deployment device and a needle-dilator assembly for placing a helical suture in myocardial tissue to access a left ventricle in accordance with the methods of the present invention.

### DETAILED DESCRIPTION OF THE INVENTION

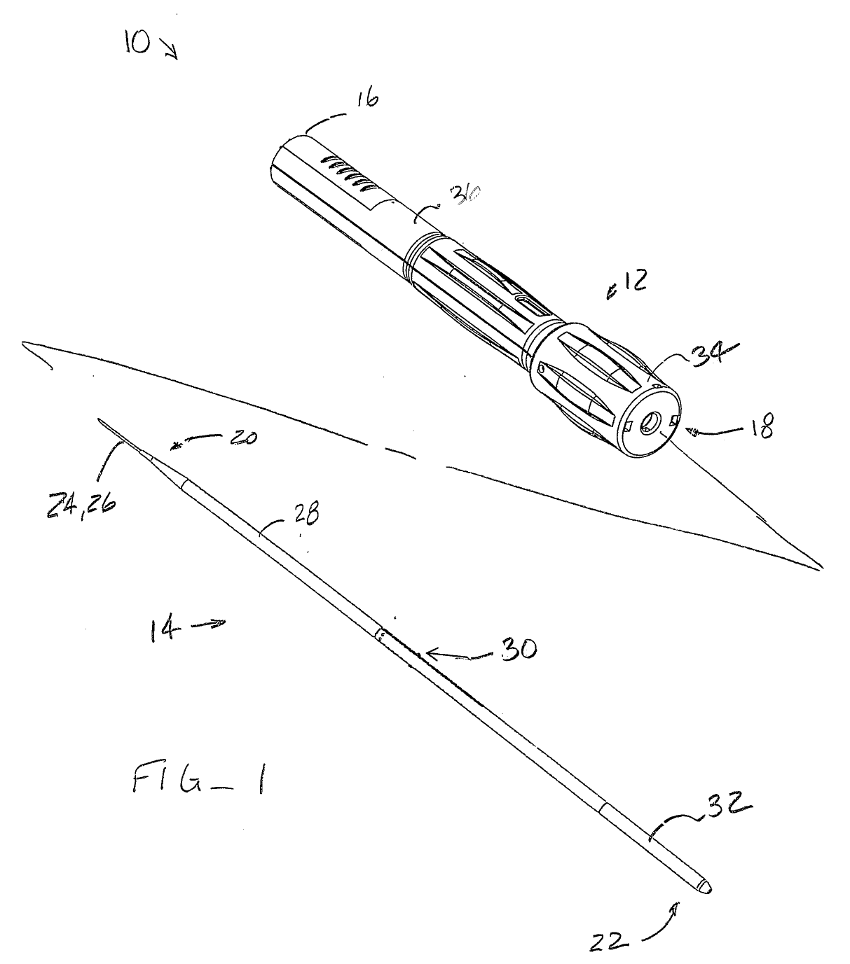

Referring now to FIGS. 1-3, a system for helically advancing suture through tissue 10 comprises a suture deployment device 12 and a needle-dilator assembly 14. The suture deployment device 12 comprises a handle 36 having a distal end 16, a proximal end 18, and a central passage. A knob 34 is rotatably mounted at a proximal end 22 of the handle, as will be described in more detail, and a straight needle 24 is slidably mounted in a central passage of a dilator body 28. The straight needle is initially immobilized relative to the handle of the suture deployment device 12 by a latch element 30, as will be described in more detail below. A distal tip 26 of the needle 24 extends distally of the dilator body 28, typically by short distance in the lane from 1 cm to 3 cm, typically being about 1.5 cm, when the needle is being held within the handle by the latch.

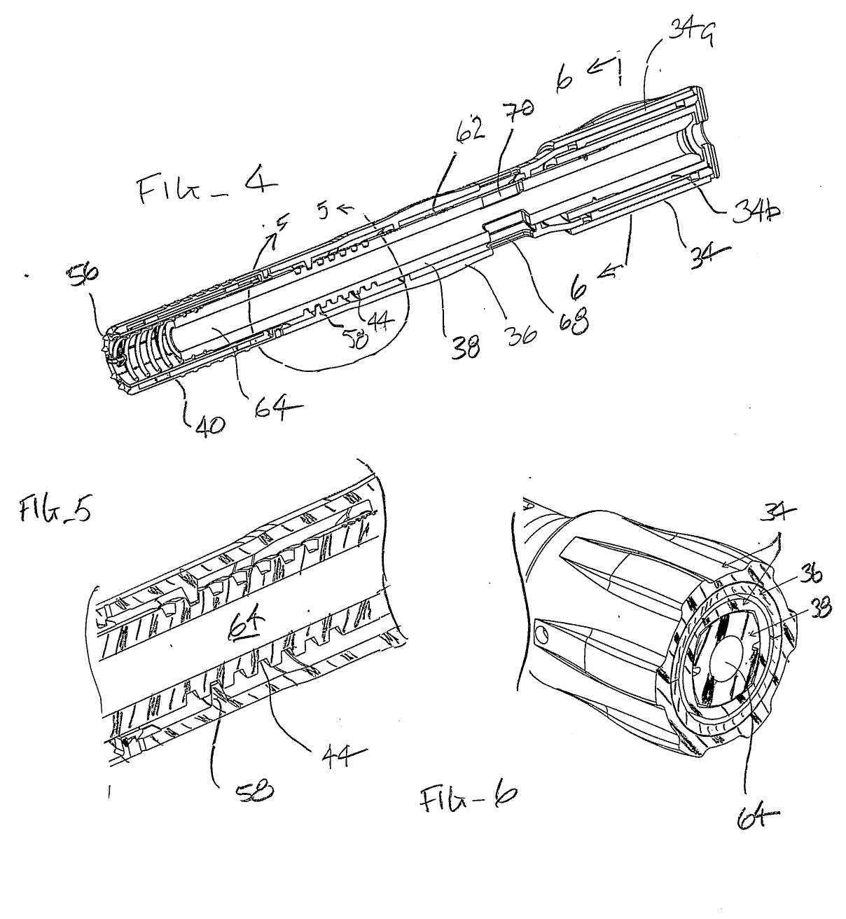

Referring now in particular to FIG. 2, the suture deployment device 12 further includes a shuttle member 38 which is mounted within a central passage 62 of the handle 36. Conveniently, the handle 36 is formed from two C-shaped shells 36a and 36b to facilitate assembly. A pair of helical needles 40 are attached to helical grooves 42 on the distal end of the shuttle member 38. Typically, the helical needles will be mounted so that the turns of the needle are interlaced and the needles are 180° out-of-phase. A helical track 44 is formed on an outer surface of the shuttle member 38, and the helical track engages a follower 58 (FIG. 5) so that rotation of the shuttle member will cause the shuttle member to axially translate to deploy or retract (depending on the direction of roatation) the helical needles 40. One receptacles 46 is formed on each side of the handle 36, and each receptacle configured to receive a suture length 82 attached to each of the helical needles 40. Receptacles are enclosed by removable covers 48, and the suture lengths 82 will be deployed from the receptacles after the needles have been implanted in tissue and the shuttle deployment device 12 is withdrawn from over the tissue.

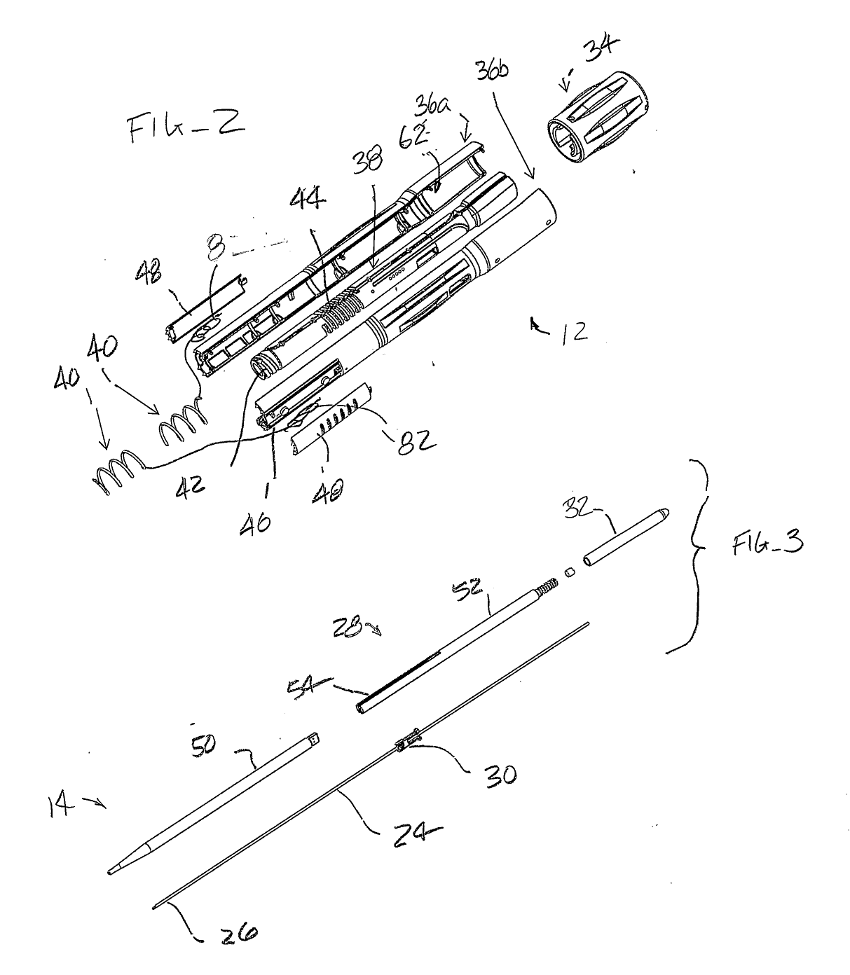

With reference to FIG. 3, the dilator body 28 of the needle-dilator assembly 14 has a distal portion 50, a middle portion 52, and the flashback chamber 32 at a proximal end. A slot 54 is formed in the middle portion which receives the latch member 30 as described in more detail below.

Referring now to FIGS. 4-6, engagement between the helical track 44 and the follower 58 formed on the inside surface of the handle can be better seen. The shuttle member 38 can be rotated by the knob 34, as will be described below, and such rotation will cause the helical track to advance over the follower 58 which is fixed to the handle 36. The knob 34, in turn, is coupled to a proximal portion of the shuttle member 38. The handle includes an outer shell 34a and a coaxial inner shell 34b so that manual rotation of the outer shell will cause the inter shell to rotate in a like manner The inner shell 34b, in turn, has a pin or other feature (not shown) which engages and travels within an axial slot or similar channels or track formed on an outer surface of a proximal portion of the shuttle member 38. In this way, rotation of the knob 34 is imparted to the shuttle member 38, causing the shuttle member to both rotate and axially translate, but without requiring the knob to translate or change axial position relative to the handle. The concentric nature of the knob 34, handle 36, and shuttle member 38 is best seen in FIG. 6. FIG. 6 also illustrates a central passage 64 of the shuttle which extends the entire length of the shuttle member and provides an access path for the needle-dilator assembly 14.

Windows 68 and 70 are formed, respectively, in the handle 36 and the shuttle member 38 to allow user to observe the needle-dilator assembly 14 within the passage 64 as well as to observe the rotation and advancement of the shuttle member 38 during a procedure.

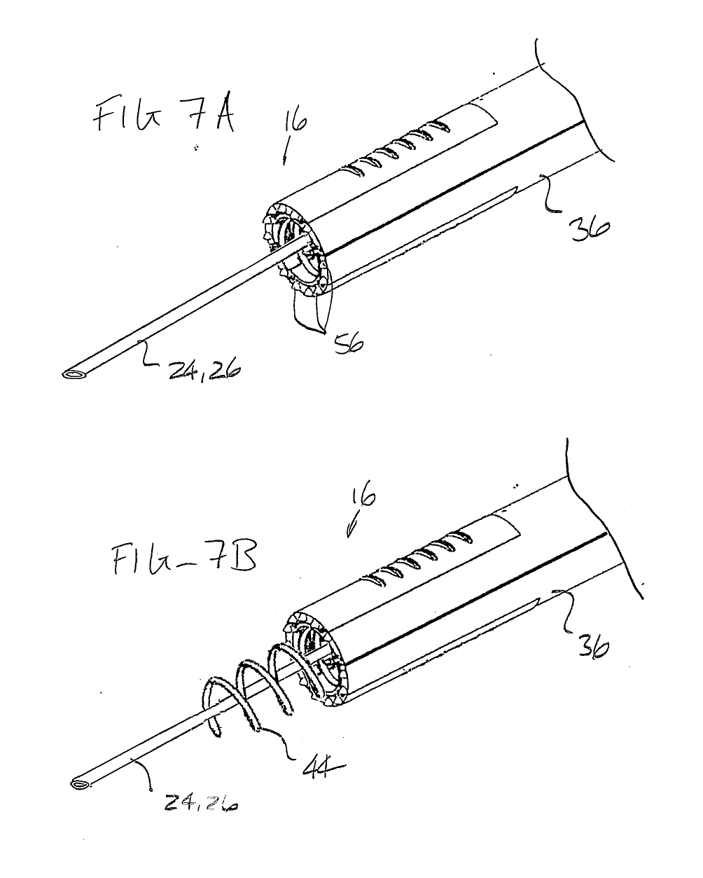

Referring now to FIG. 7A and 7B, the distal tip 26 of the straight needle 24 will extend distally from the distal end 16 of the suture deployment device 12, as shown in FIG. 7A, at the beginning of a procedure. The distal tip 26 of the needle 24 extends sufficiently beyond the distal end of the handle 36 so that the needle can be advanced through the thickness of a typical myocardium near the apical region of a patient's heart, typically being 1 cm to 3 cm. The distal tip of the handle 36 will also typically include cleats 56 which can engage and stabilize against the epicardium of the heart after the straight needle 24 is penetrated through the myocardium. As shown in FIG. 7B, the helical needle(s) 44 may be advanced from the handle 36 coaxially over the staright needle 24 and into tissue after the needle has been penetrated into that tissue.

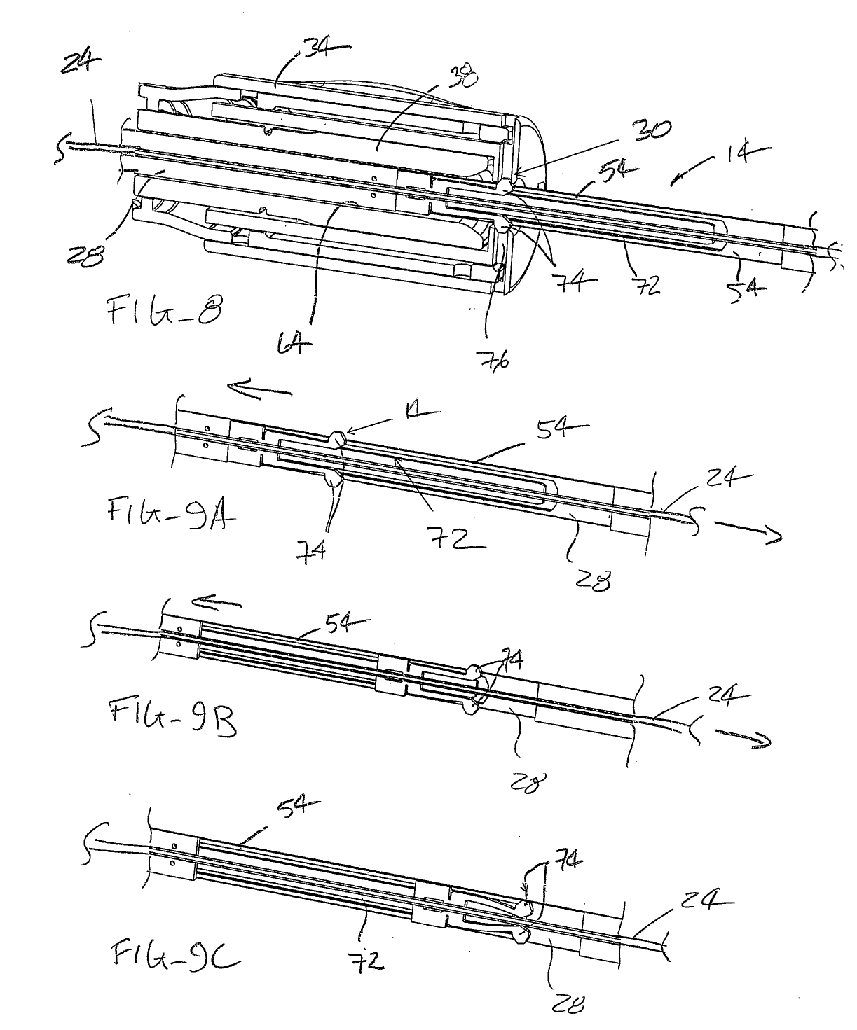

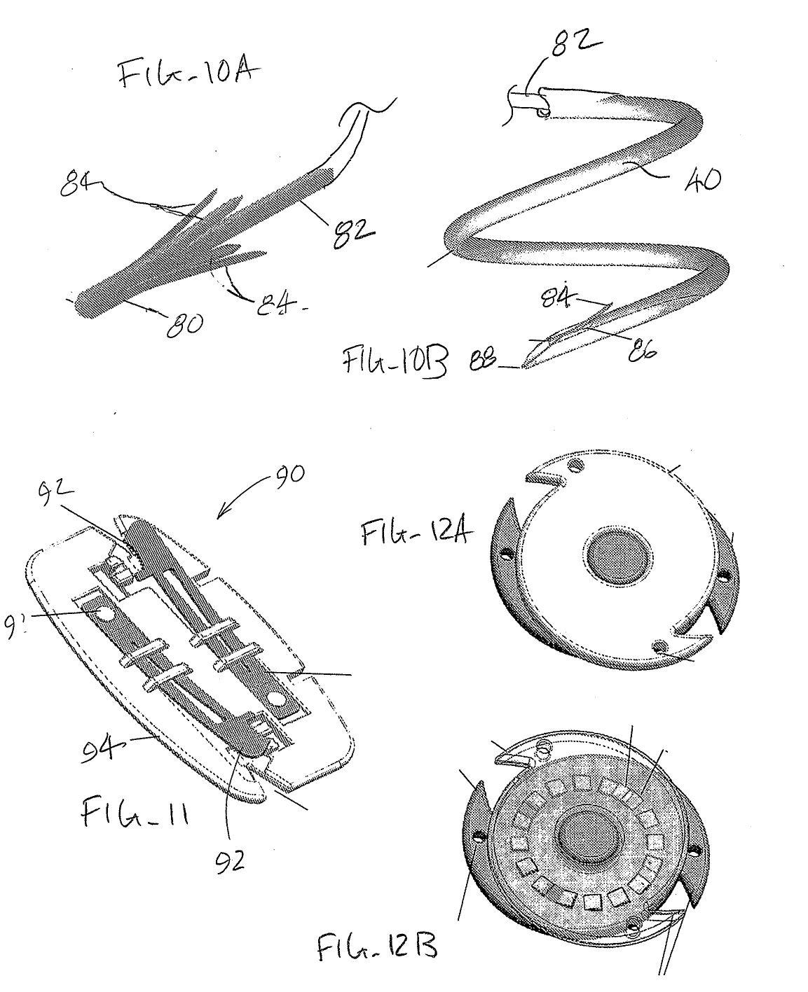

Referring now to FIGS. 8 and 9A-9C, at the beginning of a procedure, the needle-dilator assembly 14 will be present within the central passage 64 of the shuttle member 38 and will be held in position by the latch 30. In particular, the latch member 30 includes a pair of detents 74 which engage a groove 76 formed and a proximal end of the handle. The latch 30 is connected directly to the straight needle 24 so that the needle will not move until the latch is disengaged. The dilator body 28 includes a slot 54 which allows the dilator body to be advanced over the needle by accommodating the detents 74. The detents 74, however, are spring loaded to close radially inwardly (being attached at the ends of a spring-like U-shaped frame), but are held open by a cam member or track 72 which is coupled to move with the dilator body. As seen in FIG. 9A, dilator body 28 is initially retracted fully proximally relative to the needle 24 so that the detents are held fully open by the cam member 72. As the dilator body 28 is advanced distally relative to the needle, however, the latch member travels to the right, as shown in FIGS. 9B and 9C, until the cam member ends and the latch frame springs closed to radially retract and close the detents 74, as shown in FIG. 9C. Once the detents closed, they fall out of engagement with the groove in the handle so that the needle is now free to move together with the dilator body without attachment to the handle. The dilator body 28, however, will have been advanced fully to cover the needle 24 so that the needle is protected against accidental tissue penetration.

Referring now to FIGS. 10A and 10B, an exemplary tissue anchor 80 may be attached to a length of suture 82 with a plurality of self-deploying arms or barbs 84. The self-deploying arms or barbs 84 diverge radially in a proximal direction so that the anchor and suture may advance distally through tissue with minimal force but will embed in the tissue with a significant retention force when retracted in a proximal direction. Distal tips 88 of the helical needles 40 have a slot 86 that allows a single arm or barb 84 of the tissue anchor 80 project outside of the helical needle while the other arms remain fully contained inside the helical needle. The free tissue anchor arm 84 flexes down as the helical needle is driven through tissue distally, and when rotation of the helical needle is reversed to retract the needle, the exposed arm 84 penetrates into the tissue and the anchor 80 deploys in place. Once released from the helical needle, the remaining tissue anchor arms 84 similarly embed into the tissue and provide additional retention. Other anchor designs might also find use, such as a T-bar design, adhesive based design, barbed prongs, and the like. Usually, the anchor will be embedded into tissue, but at the distal end of the suture may be anchored within a heart chamber or other open body cavity or may exit the walls of the tissue to another external surface such that they may be secured without the use of anchors.

In some embodiments the device may be configured such that the anchor at the distal end of the suture can be advanced from the tip of the helical needle to a position exterior of the heart. For example, the anchor may be a sharp rod or tube formed from a shape memory alloy where the rod or tube can be advanced from the distal end of the helical needle. As it advances, the shape memory may direct the anchor tube towards the central axis defined by the dilator. The anchor may then engage and be captured by the dilator so that removal of the dilator in a subsequent subsequent step will withdraw a free end on the suture which creates a “looped” suture to affix the suture end in the tissue without the implantation of an anchor.

In still other embodiments, the anchor described above may be configured to exit the heart tissue as it is advanced. In still other embodiments, the device may include retrieval components which are configured to enter the heart tissue when the helical needles are fully advanced and then align and engage with the anchor such that removing the retrieval features pulls the anchors out of the heart tissue. Still other devices and methods for creating a looped suture path within the heart tissue may also be employed within the principles of the present invention.

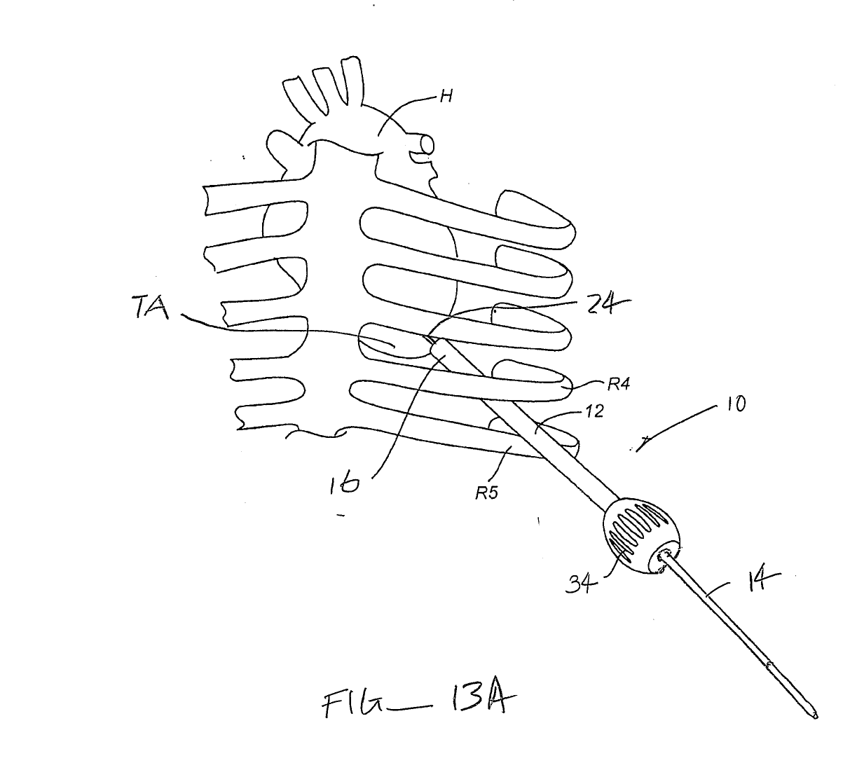

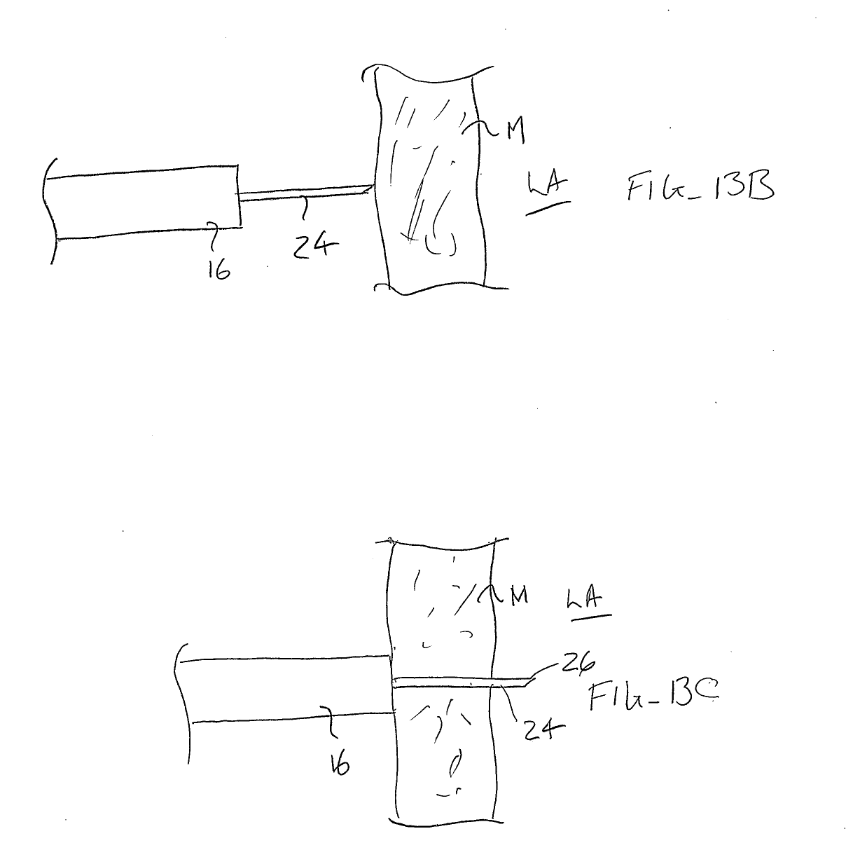

Referring now to FIGS. 11, 12A and 12B, a free end of the suture on an outer tissue surface overlying the tissue tract to be closed may be cinched and “tied-off” using a pledget which provides “auto-locking” of the suture via a mechanism which allows passage of suture through the pledget in one direction but not the opposite direction. Use of a pledget is advantageous as the pledget will hold the sutures in place after placement without the need to tie a knot. Additionally, the pledget maintains tension that is normal to the tissue plane to minimize the effects of “cheese-wiring” which is the tendency of unsupported suture to cut through tissue.

As shown in FIG. 11, a first embodiment of a pledget 90 has arms 92 typically made of stainless steel which are allowed to flex away from but not toward a base 94. This allows the pledget 90 to slide down suture material easily, while the arms are free to flex out, but prevents the pledget from moving in the opposite direction, as the arms are secured flat with the pledget base. The pledget base may be constructed of a single component or multiple components to allow greater conformance to the topology of the heart Finally, the pledget may be pre-applied to the tip of the delivery device to allow simultaneous deployment of the helices/ sutures and pledget or subsequently applied after the insertion of the port, prior to its removal. After completion of the surgery, pledget(s) may be placed over the sutures and the sutures are tensioned until hemostasis is reached.

As shown in FIGS. 12A and 12B, a pledget 100 comprises a head disc 102 rotatably attached to a base disc 104. The discs define opposed pinching arms 106 which define closing slots which pinch and hold the suture in place. Spurs and pinions 108 and 110 prevent the discs from rotating out of position. Suture is placed between the suture pinching arms and gripping features, such as holes 112, for a locating and placement tool are used to rotate the discs such that the suture pinching arms tightly and securely grasp the suture.

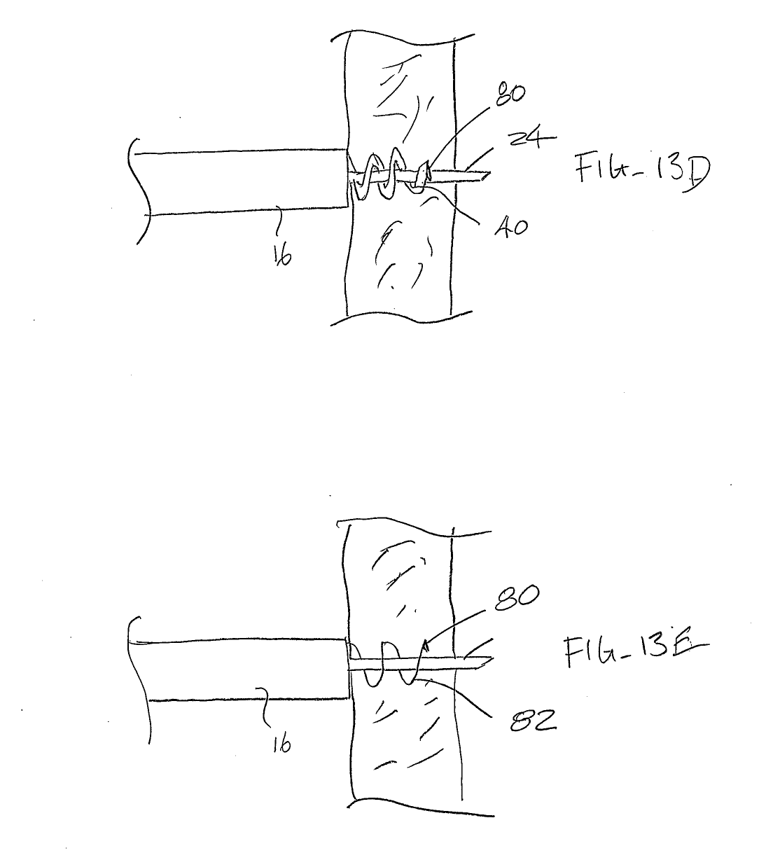

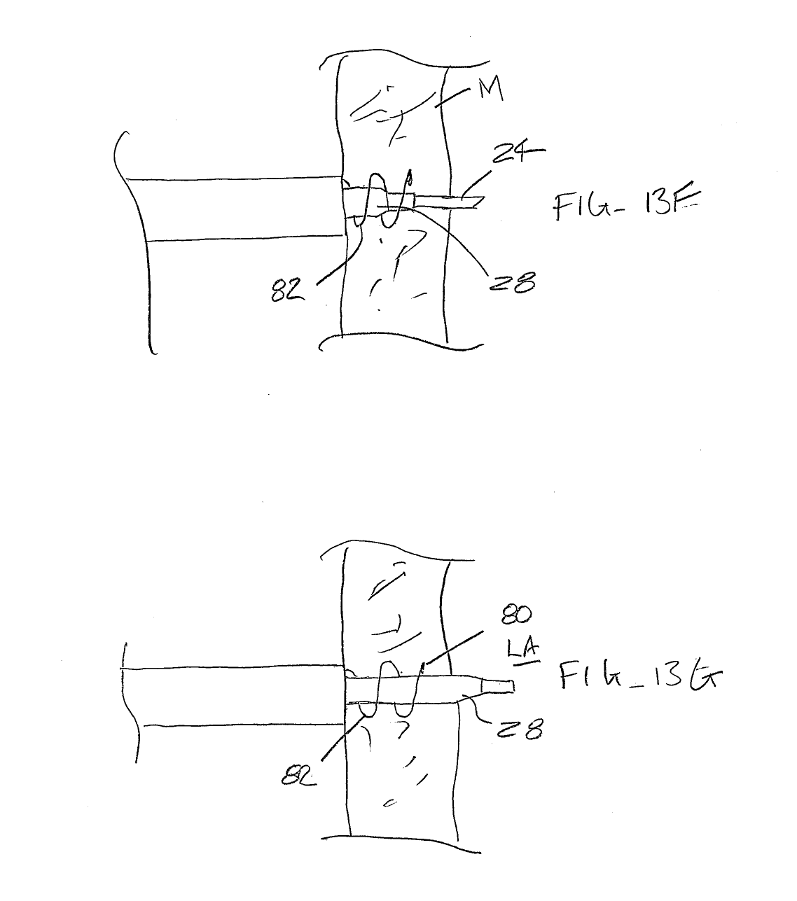

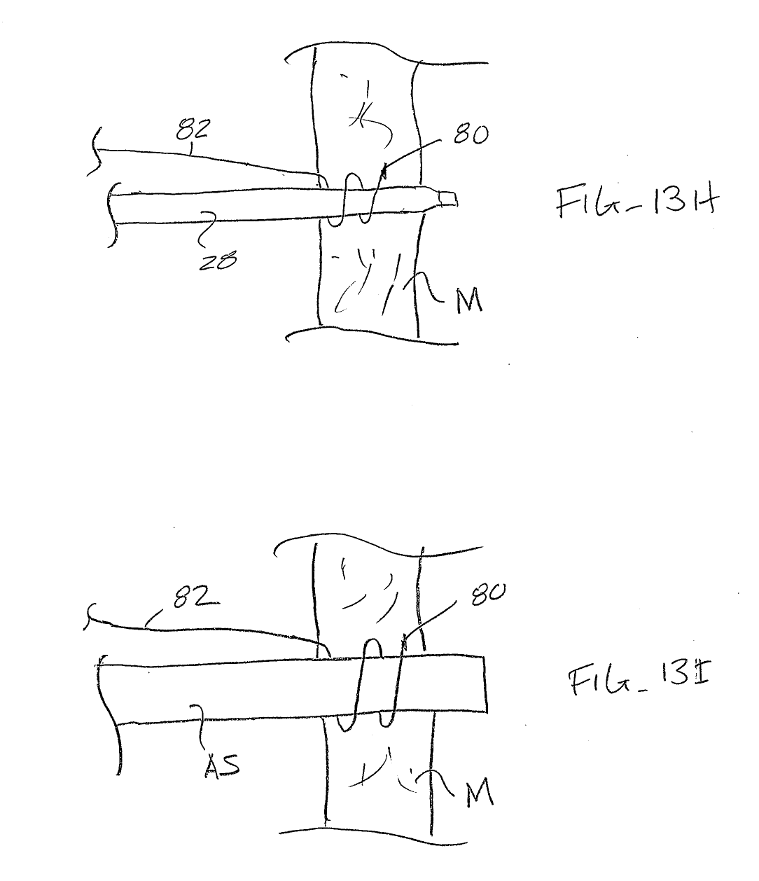

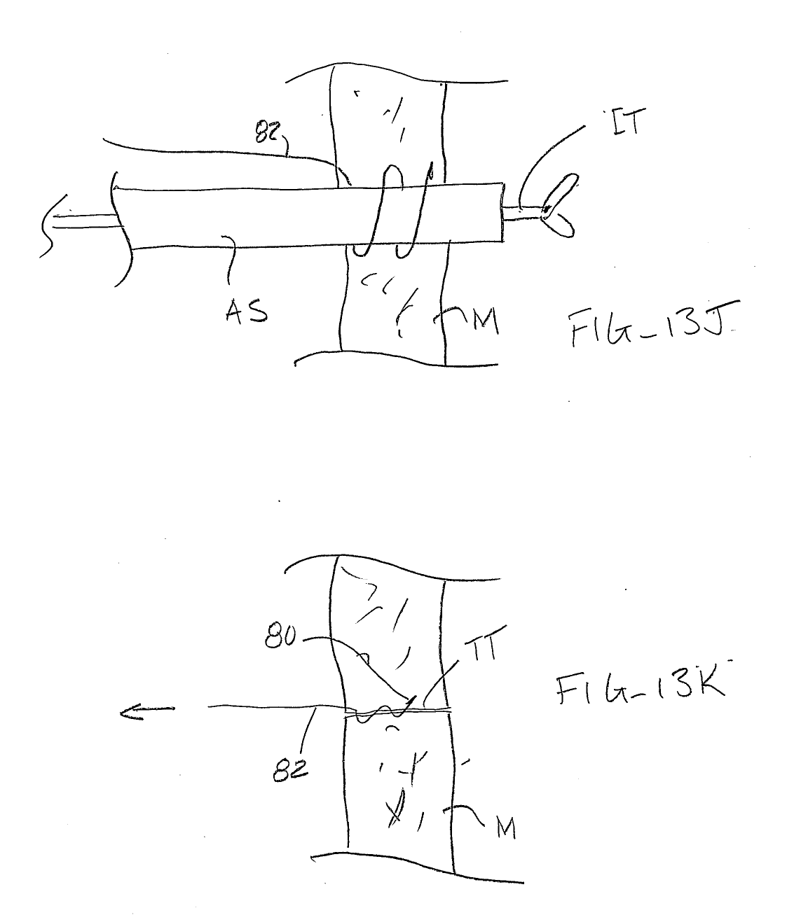

Referring now to FIGS. 13A-13K, an exemplary protocol for transapically accessing a left ventricle or other heart chamber and performing an intra cardiac procedure according to the principles of the present invention will be described. The relevant anatomy is illustrated in FIG. 8A where a transapical region TA of a patient's heart H is protected behind the patient's ribs. Access will generally be acquired through an intercostal space between rib R4 and rib R5. The distal end 16 of the suture deployment device 12 is introduced between the ribs so that straight needle 24 can engage the transapical region TA of the heart H.

Referring now to FIGS. 13B and 13C, the needle 24 may be advanced through the myocardium M so that the distal end 26 of the needle enters the left atrium LA. At this point, blood will enter the hollow needle 24 and flow back to the flashback chamber 32, allowing the physician to confirm that that the needle has entered the left atrium.

After needle entry is confirmed, the knob 34 can be rotated to rotate and advance the helical needles 40 (only one of which is shown in FIG. 13D) into tissue. After extending the needles by a desired distance into the myocardium, and typically not into the left atrium, the rotation of the needles 40 will be reversed, leaving the suture 82 in place. Typically, an anchor 80 at the distal end of the suture 82 will seal-deploy, anchoring a distal end of the suture in place so that the length of suture assumes a helical pattern as the needle is withdrawn.

After the suture is properly deployed, the dilator body 28 may be advanced over the needle 24 and into the left atrium LA, as shown in FIGS. 13F and 13G. Note that although the dilator body 28 is advanced, the needle does not further advance because of the latching mechanism 30 described above. Thus, the dilator body will advance fully over the needle, as shown in FIG. 13G.

After the dilator body 28 has been fully advanced, the suture deployment device 12 may be withdrawn, leaving the dilator in place as shown in FIG. 13H. An access sheath AS may then be deployed over the dilator body 28, as shown in FIG. 13I. The access sheath will allow the introduction of interventional tools IT, as shown in FIG. 13J, so that various interventional procedures may be performed, including those listed above. After the interventional procedures are performed, the interventional tools and access sheath will be withdrawn and the suture length 82 pulled in a proximal direction in order to cinch the tissue tract TT, as shown in FIG. 13K. The anchor 80 will remain in the tissue and allow the suture to be cinched in order to achieve the desired closure.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A system for helically advancing suture through tissue, said system comprising:
a handle having a distal end, a proximal end, and a central passage extending between said ends;
a knob rotatably carried on the proximal end of the handle;
a shuttle member reciprocatably disposed in the central passage of the handle, said shuttle member having a distal end, a proximal end, and a central passage extending between said ends;
at least one helical needle coupled to the distal end of the shuttle member; and
suture releasably carried by the at least one helical needle;
wherein the knob is coupled to the shuttle member so that the rotation of the knob rotates and axially translates the shuttle member to rotate and translate the at least one helical needle, wherein the knob does not axially translate as it is rotated.

2. A system for helically advancing suture through tissue as in claim 1, further comprising a needle-dilator assembly adapted to be received through a central passage in the shuttle member.

3. A system for helically advancing suture through tissue as in claim 2, wherein the distal end of the handle is adapted to be engage a pericardial or myocardial surface of a patient's heart.

4. A system for helically advancing suture through tissue as in claim 2, wherein the handle is adapted to access the pericardial or myocardial surface of an apical region of the heart through an intercostal access site.

5. A system for helically advancing suture through tissue as in claim 2, wherein the handle is adapted to access the pericardial or myocardial surface of an apical region of the heart through a subxiphoid approach.

6. A system for helically advancing suture through tissue as in claim 1, wherein the at least one helical needle is fixedly attached to the distal end of the shuttle member so that the needle is advanced through tissue as the shuttle member rotates and advances.

7. A system for helically advancing suture through tissue as in claim 1, wherein the at least one helical needle is hollow and the suture is received in the needle.

8. A system for helically advancing suture through tissue as in claim 1, wherein the handle has at least one pocket disposed on an outer surface near the distal and the suture is received in the pocket.

9. A system for helically advancing suture through tissue as in claim 1, wherein the suture carries barbs along a distal region and wherein the barbs are adapted to self-deploy to anchor in myocardial tissue as the helical needle is withdrawn.

10. A system for helically advancing suture through tissue as in claim 1, wherein the suture carries one or more anchors along a distal region and wherein the anchors are adapted to self-deploy within an open tissue chamber as the helical needle is withdrawn.

11. A system for helically advancing suture through tissue as in claim 1, wherein an outer surface of the shuttle member and an inner surface of the central passage of the handle together define a helical track and a track follower so that rotation of the shuttle member relative to the handle cases the shuttle member to axially translate relative to the handle.

12. A system for helically advancing suture through tissue as in claim 11, further comprising a coupling element on the knob which engages a coupling element on the shuttle member so that rotation of the knob is transferred to the shuttle member while allowing the shuttle member to axially translate in response to interaction of the helical track and the track follower.

13. A system for helically advancing suture through tissue as in claim 2, wherein the needle-dilator assembly is pre-mounted in the central passage in the shuttle member in a packaged configuration.

14. A system for helically advancing suture through tissue as in claim 13, wherein a needle of the needle-dilator assembly extends distally from a distal end of the central passage in the shuttle member while a dilator body of the needle-dilator assembly remains retracted within of the central passage in the shuttle member in the packaged configuration .

15. A system for helically advancing suture through tissue as in claim 15, wherein the needle of the needle-dilator assembly is latched to the handle so that the dilator body of the needle-dilator assembly may be advanced over the needle without advancing the needle, wherein the needle unlatches when the dilator is fully advanced over the needle so that the needle can be withdrawn from the shuttle member.

16. A system for helically advancing suture through tissue as in claim 15, wherein the needle of the needle-dilator assembly has a spring-loaded detent which travels over a cam surface of the dilator body, wherein the detent engages the handle in the packaged configuration and wherein the dilator body is configured to be advanced to reposition the cam surface to allow the detent to fall out of engagement with handle, allowing the needle to be withdrawn from the shuttle member after the needle is covered by the dilator body.

17. A method for transapical access to a heart chamber, said method comprising:
positioning a distal end of a handle against an apex of a patient's heart to advance a distal end of a straight needle through myocardial tissue into a heart chamber;
rotating and advancing at least one helical needle from the distal end of the handle into the myocardial tissue surrounding the needle, wherein the at least one helical needle carries a length of suture;
reverse rotating and retracting the at least one helical needle surrounding the straight needle to embed the length of suture in a helical path surrounding the straight needle in the myocardial tissue;
advancing a dilator from the handle over the straight needle within the embedded helical suture, wherein the straight needle is latched to the handle so that the needle cannot advance further into the heart chamber as the dilator advances and wherein the needle is unlatched from the handle when the dilator fully cover the needle;
removing the handle over the dilator;
advancing an access sheath over the dilator to provide interventional access into the heart chamber.

18. A method for transapical access to a heart chamber as in claim 17, further comprising observing blood flashback through the needle to confirm entry of the needle into the heart chamber.

19. A method for transapical access to a heart chamber, wherein the needle carries a spring-loaded detent that engages the handle, wherein advancement of the dilator fully over the needle disengages the detent from the handle to allow the needle to move freely of the handle.

20. A method for transapical access to a heart chamber, wherein rotating and advancing the at least one helical needle comprises rotating a knob relative to the handle, wherein the knob is coupled to a shuttle member that carries the at least one helical needle, wherein rotation of the knob rotates and axially translates the shuttle member to rotate and translate the at least one helical needle, wherein the knob does not axially translate as it is rotated.

21. A method as in claim 17, wherein all methods steps are performed while the heart is beating.

22. A method as in claim 17, wherein tension is maintained on the pericardium to stabilize the heart while the helical needle is being advanced.

23. A method as in claim 22, wherein the helical needle is first passed through the pericardium surrounding the heart, the helical needle is then drawn proximally to tension the pericardium and stabilize the heart, and the helical needle is advanced into the myocardium while the tension is maintained on the pericardium.

24. A method as in claim 17, wherein the helical needle is first positioned adjacent the apical region of the heart via an intercostal approach.

25. A method as in claim 17, wherein the needle is first positioned adjacent the apical region of the heart via an subxiphoid approach.

26. A method as in claim 17, wherein advancing comprises advancing two or more helical needles simultaneously to position two or more helical sutures.

27. A method as in claim 26, wherein the two or more helical needles are located in a common cylindrical envelope.

28. A method as in claim 17, wherein a distal portion of the suture has self-deploying barbs which anchor when the suture is tensioned proximally.

29. A method as in claim 17, wherein a distal end of the suture is anchored in the heart chamber.

30. A method for performing a cardiac procedure, said method comprising:
accessing the heart chamber as in claim 17;
introducing at least one tool through the dilated passage while the helical suture remains in place;
performing the cardiac procedure with the at least one tool; removing the at least one tool from the dilated passage; and
drawing on the suture to close the dilated passage.

31. A method as in claim 30, wherein the cardiac procedure comprises valve replacement.

32. A method as in claim 30, wherein the cardiac procedure comprises valve repair.

33. A method as in claim 30, wherein the cardiac procedure comprises left atrial appendage closure.

34. A method as in claim 30, wherein the cardiac procedure comprises cardiac ablation.

35. A method as in claim 30, wherein the cardiac procedure comprises closure of an atrial septal defect.

36. A method as in claim 30, wherein the cardiac procedure comprises closure of a patent foramen ovale.

37. A method as in claim 30, wherein the cardiac procedure comprising aneurysmectomy.
